(19) United States
(12) Patent Application Publication
(10) Pub. No.: US 2011/0082480 A1
(43) Pub. Date: Apr. 7, 2011

(54) WOUND CLOSURE DEVICE INCLUDING PIVOTABLE CLAWS

(75) Inventor: Frank J. Viola, Sandy Hook, CT (US)

(73) Assignee: TYCO HEALTHCARE GROUP LP, New Haven, CT (US)

(21) Appl. No.: 12/887,890

(22) Filed: Sep. 22, 2010

Related U.S. Application Data
(60) Provisional application No. 61/247,639, filed on Oct. 1, 2009.

(51) Int. Cl. A61B 17/03

(52) U.S. Cl. 606/151

(57) ABSTRACT

A wound closure device includes a shaft and a claw assembly. The shaft defines a longitudinal axis. The claw assembly is operably associated with the shaft and includes a housing and one or more one claws pivotally mounted to the housing and positionable between a first position and a second position in which the one or more claws are lockingly engaged with tissue adjacent a wound for maintaining the wound in a closed position to promote healing thereof. The shaft is adapted to separate from the claw assembly in the second position.

### CROSS REFERENCE TO RELATED APPLICATION

The present application claims the benefit of and priority to U.S. Provisional Application Ser. No. 61/247,639 filed on Oct. 1, 2009, the entire contents of which are incorporated herein by reference.

### BACKGROUND

1. Technical Field

The present disclosure relates to a wound closure device, and more particularly, to a wound closure device having pivotable claws for closing a wound.

2. Description of Related Art

Puncture wounds, wounds that pierce through tissue, may result from trauma or may be intentionally created in order to provide access to a body cavity during surgical procedures. During endoscopic surgical procedures, for example, a trocar device is utilized to puncture the peritoneum to provide an access port by way of a cannula through the abdominal wall. Generally, a trocar and/or a cannula is placed through the abdominal wall for introduction of surgical instrumentation which is necessary to carry out the surgical procedure. In this manner, the surgeon may introduce a surgical instrument such as a grasper, scissor, clip applier, stapler or any other surgical instrument which may be necessary during the particular surgical procedure. Once the procedure is complete, it is necessary to close the wound in order to protect against hernia, adhesions, and other undesirable conditions.

### SUMMARY

Accordingly, a wound closure device includes a shaft and a claw assembly. The shaft defines a longitudinal axis. The claw assembly is operably associated with the shaft and includes a housing and one or more claws pivotally mounted to the housing. The one or more claws are positionable between a first position and a second position in which the one or more claws are lockingly engaged with tissue adjacent a wound for maintaining the wound in a closed position to promote healing thereof. The shaft may be adapted to separate from the claw assembly in the second position. The shaft may include a frangible portion adapted to fracture in the second position.

In embodiments, one or more cams are supported for rotation on the distal end of the shaft. The one or more cams rotate the one or more claws between the first and second positions. The one or more cams rotate in response to rotation of the shaft. The one or more cams may include one or more notches adapted to lock the one or more claws in the second position when the one or more claws are engaged thereto.

In embodiments, the claw assembly includes a plurality of claws. In embodiments, the plurality of claws is pivotably mounted to a bridge disposed therebetween. The shaft may be disposed in mechanical cooperation with the claw assembly such that the shaft separates from the claw assembly upon the mechanical disengagement therefrom. In embodiments, one or both of the claw assembly and the shaft are biodegradable.

In one embodiment, one or more linkages are operably associated with the one or more claws and the housing. The one or more linkages may be living hinges. The one or more linkages are operably coupled to one or both of the housing and the one or more claws by one or more pivot pins. In embodiments, the housing axially translates along the longitudinal axis of the shaft when the one or more claws move between the first and second positions. The housing may be locked in a proximal-most position when the one or more claws are in the second position.

In one aspect, a method of closing a wound includes providing a wound closure device having a shaft and a claw assembly including a housing and one or more claws pivotally mounted to the housing. The method includes inserting the one or more claws into tissue adjacent an open wound; actuating the wound closure device such that the one or more claws close the wound upon the pivotal movement of the one or more claws; and detaching the shaft from the claw assembly. The method includes removing at least a portion of the shaft from the wound. The method may include rotating the shaft to pivot the at least one claw. In embodiments, the method includes pulling the shaft proximally to pivot the one or more claws. The method may include fracturing a frangible portion of the shaft.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a perspective view of one embodiment of a wound closure device in a first position in accordance with the present disclosure;

FIG. 2 is a perspective view of the wound closure device of FIG. 1 in a second position;

FIG. 3 is a perspective view of another embodiment of a wound closure device in a first position in accordance with the present disclosure; and

FIG. 4 is a perspective view of the wound closure device of FIG. 3 in a second position.

### DETAILED DESCRIPTION

Particular embodiments of the present disclosure will be described herein with reference to the accompanying drawings. As shown in the drawings and as described throughout the following description, and as is traditional when referring to relative positioning on an object, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is farther from the clinician during proper use. In the following description, well-known functions or constructions are not described in detail to avoid obscuring the present disclosure in unnecessary detail.

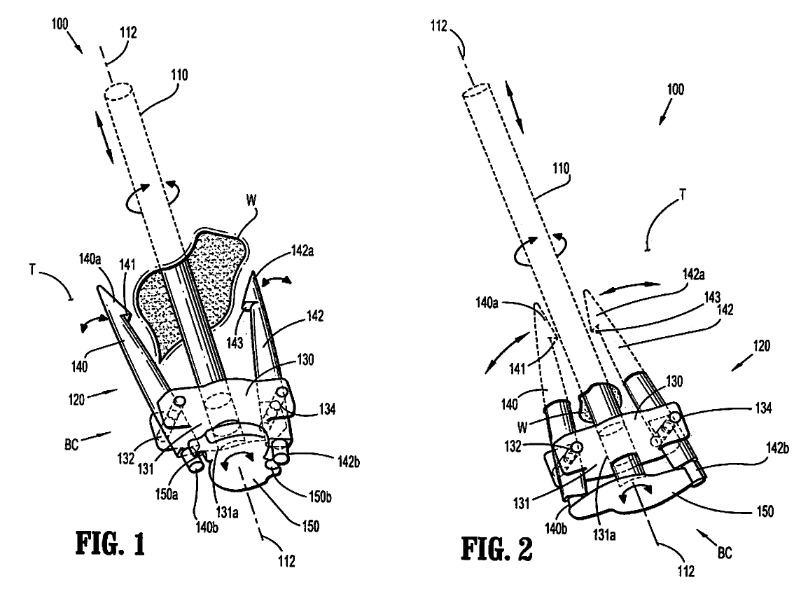

Referring now in specific detail to the drawings, in which like numbers identify similar or identical elements, FIGS. 1 and 2 illustrate an embodiment of a wound closure device and is generally designated 100. The wound closure device 100 includes a shaft 110 and a claw assembly 120. The claw assembly 120 is operably associated with the shaft 110 and includes a housing 130, claws 140, 142, and a cam 150. The housing 130 includes a housing body 131 and pivot pins 132, 134. The housing 130 is mounted to the distal end of the shaft 110 via shaft bore 131a extending through the housing 130 along a longitudinal axis 112 of the shaft 110. The claws 140, 142 are pivotally mounted to the housing 130 via pivot pins 132, 134 and include tips 140a, 142a at the proximal ends thereof for engaging tissue “T” and extensions 140b, 142b at the distal end thereof. The tips 140a, 142a have one or more barbs 141, 143 extending therefrom. The extensions 140b, 142b are adapted to lockingly engage the cam 150 as described below. The claws 140, 142 are positionable between a first position (FIG. 1) and a second position in which the claws 140, 142 are lockingly engaged with the tissue “T” adjacent a wound “W” for maintaining the wound “W” in a closed position (FIG. 2). The cam 150 is supported for rotation on the distal end of the shaft 110. The cam 150 rotates in response to rotation of the shaft 110. As the cam 150 rotates, the claws 140, 142 pivot between the first and second positions. The cam 150 may include one or more notches 150a, 150b adapted to engage the extensions 140b, 142b of the claws 140, 142 and are adapted to lock each claw 140, 142 in the second position when the extensions 140b, 142b of the claws 140, 142 are engaged with the one or more notches 150a, 150b.

The shaft 110 may be adapted to separate from the claw assembly 120 in the second position such that the claw assembly 120 remains attached to the tissue “T” and within the body cavity “BC” so that the wound “W” may remain closed for effective healing thereof. The shaft 110 may include a frangible portion (see FIG. 4) adapted to fracture in the second position. In embodiments, a portion of the shaft 110 and/or the claw assembly 120 is biodegradable, allowing the wound “W” to heal as they are absorbed into the body. Also, the shaft 110 may be formed of absorbable material and cut near the surface of skin.

In use, the claw assembly 120 is inserted into the wound “W” such that the claws 140, 142 are disposed within a body cavity “BC” of a patient. The claws 140, 142 are then positioned within internal tissue “T” adjacent the wound “W” by proximally translating the shaft 110 such that each claw 140, 142 is fixedly engaged within the tissue “T.” Upon rotation of the shaft 110, the cam 150 rotates each claw 140, 142 between the first and the second position. The notches 150a, 150b of the cam 150 engage extensions 140b, 142b of the claws 140, 142 such that the claws 140, 142 are lockingly engaged in the second position. The shaft 110 may then be removed from the claw assembly 120 and out from the wound “W.” The shaft 110 may be mechanically disengaged (e.g., unscrewed) for removal thereof from the claw assembly 120. In embodiments, as discussed above, the shaft may have a frangible portion adapted to fracture upon the application of force in order to remove a portion of the shaft proximal the frangible portion (see FIG. 4) from the claw assembly 120.

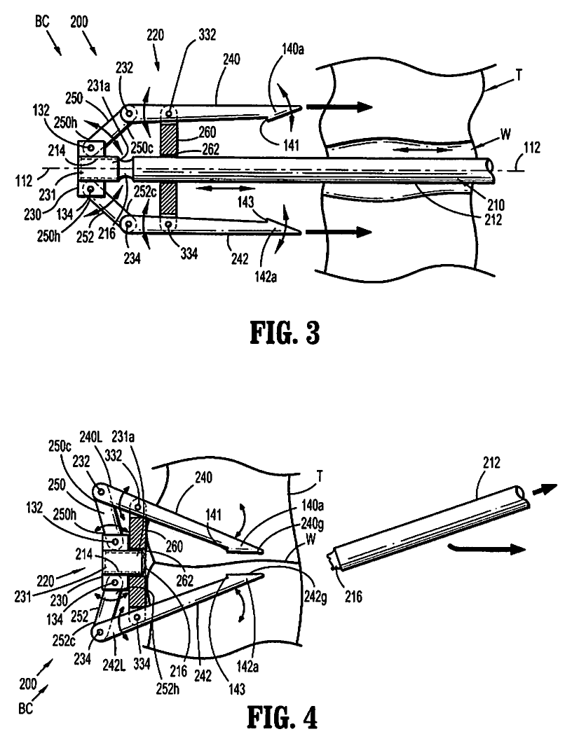

Referring now to FIGS. 3 and 4, another embodiment of a wound disclosure device 200 includes a shaft 210 and a claw assembly 220. The shaft 210 includes a proximal portion 212, a distal portion 214, and a frangible portion 216 sandwiched therebetween. The claw assembly 220 is operably associated with shaft 210 and includes a housing 230, claws 240, 242, linkages 250, 252, and a bridge 260.

With continued reference to FIGS. 3 and 4, the housing 230 includes a housing body 231 and pivot pins 132, 134. The housing 230 is mounted to the distal end of the shaft 210 via a shaft bore 231a extending through the housing 230 along a longitudinal axis 112 of the shaft 210. The claws 240, 242 are pivotally mounted to the linkages 250, 252 via pivot pins 232, 234 and are pivotally mounted to the bridge 260 via pivot pins 332, 334. The bridge 260 includes a bore 262 therethrough for permitting passage of the shaft 210 therethrough. The claws 240, 242 include tips 140a, 142a at the proximal ends thereof for engaging tissue “T.” The tips 140a, 142a have one or more barbs 141, 143 extending therefrom. The linkages 250, 252 are pivotally mounted to the housing 230 via pivot pins 132, 134 such that the longitudinal translation of the housing 230 causes linkages 250, 252 and claws 240, 242 to move between the first and second positions. In embodiments, the linkages 250, 252 may be “living” hinges.

The claws 240, 242 are positionable between a first position (FIG. 3) and a second position in which the claws 240, 242 are lockingly engaged with the tissue “T” adjacent a wound “W” for maintaining the wound “W” in a closed position (FIG. 4). The housing 230 axially translates along the longitudinal axis 112 of the shaft 210 when the claws 240, 242 move between the first and second positions. The housing 230 may be locked in a proximal-most position (FIG. 4) when the claws 240, 242 are in the second position. In the second position, the frangible portion 216 of the shaft 210 may be at least partially disposed within the bore 262 of the bridge 260. The frangible portion 216 of the shaft 210 is configured to break or tear upon the application of a predetermined shear and/or axial force thereto. As such, the proximal portion 212 of the shaft 210 may be separated from the claw assembly 220 and out from within the wound “W” while the claw assembly 220 and the distal portion 214 of the shaft 210 remain attached to the tissue “T” and within the body cavity “BC” so that the wound “W” may remain closed for effective healing thereof. The distal portion 214 of the shaft 210 and the claw assembly 220 may be biodegradable, allowing the wound “W” to heal as they are absorbed into the body.

In use, the claw assembly 220 is inserted into the wound “W” such that the claws 240, 242 are disposed within a body cavity “BC” of a patient. The claws 240, 242 are then positioned within internal tissue “T” adjacent the wound “W” by proximally translating the shaft 210 such that each claw 240, 242 is fixedly engaged within the tissue “T.” Upon further longitudinal translation of the shaft 210, the linkages 250, 252 pivot proximally with the housing body 231 toward the bridge 260 via pivot pins 132, 134 on housing ends 250h, 252h thereof and outwardly via pivot pins 232, 234 (i.e., abaxially from the longitudinal axis 112 of the shaft 110) at claw ends 250c, 252c thereof. As the linkages 250, 252 pivot, linkage ends 240L, 242L of the claws 240, 242 pivot outwardly (i.e., abaxially) while grasper ends 240g, 242g pivot inwardly (i.e., adaxially toward the longitudinal axis 112 of the shaft 110). Accordingly, the claws 240, 242 rotate between the first and the second position in which the claws 240, 242 are lockingly engaged. The shaft 210 is then removed from the claw assembly 220 and out from the wound “W.” The shaft 210 may be mechanically disengaged (e.g. unscrewed) from the claw assembly 220 and/or by the application of axial and/or shear force to the frangible portion 216.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A wound closure device, comprising:
a shaft defining a longitudinal axis; and
a claw assembly operably associated with the shaft and including a housing and at least one claw pivotally mounted to the housing and positionable between a first position and a second position in which the at least one claw is lockingly engaged with tissue adjacent a wound for maintaining the wound in a closed position to promote healing thereof, wherein the shaft is adapted to separate from the claw assembly in the second position.

2. The wound closure device of claim 1, further comprising at least one cam supported for rotation on the distal end of the shaft.

3. The wound closure device of claim 1, wherein the at least one cam rotates the at least one claw between the first and second positions.

4. The wound closure device of claim 3, wherein the at least one cam rotates in response to rotation of the shaft.

5. The wound closure device of claim 1, wherein the at least one cam includes at least one notch adapted to lock the at least one claw in the second position when the at least one claw is engaged thereto.

6. The wound closure device of claim 1, wherein the claw assembly includes a plurality of claws.

7. The wound closure device of claim 6, wherein the plurality of claws is pivotably mounted to a bridge disposed therebetween.

8. The wound closure device of claim 1, wherein the shaft includes a frangible portion adapted to fracture in the second position.

9. The wound closure device of claim 1, wherein the shaft is disposed in mechanical cooperation with the claw assembly such that the shaft separates from the claw assembly upon the mechanical disengagement therefrom.

10. The wound closure device of claim 1, wherein at least one of the claw assembly and the shaft is biodegradable.

11. The wound closure device of claim 1, further comprising at least one linkage operably associated with the at least one claw and the housing.

12. The wound closure device of claim 11, wherein the at least one linkage is a living hinge.

13. The wound closure device of claim 11, wherein the at least one linkage is operably coupled to at least one of the housing and the at least one claw by at least one pivot pin.

14. The wound closure device of claim 1, wherein the housing axially translates along the longitudinal axis of the shaft when the at least one claw moves between the first and second positions.

15. The wound closure device of claim 14, wherein the housing is locked in a proximal-most position when the at least one claw is in the second position.

16. A method of closing a wound, comprising:
providing a wound closure device having a shaft and a claw assembly including a housing and at least one claw pivotally mounted to the housing;
inserting the at least one claw into tissue adjacent an open wound;
actuating the wound closure device such that the at least one claw closes the wound upon the pivotal movement of the at least one claw; and
detaching the shaft from the claw assembly.

17. The method of claim 16, further comprising rotating the shaft to pivot the at least one claw.

18. The method of claim 16, further comprising pulling the shaft proximally to pivot the at least one claw.

19. The method of claim 16, further comprising fracturing a frangible portion of the shaft.

20. The method of claim 16, further comprising removing at least a portion of the shaft from the wound.
